SEQUENCE LISTING (5 sequences)

SEQ ID NO: 1 - DNA, 22 bp, Artificial Sequence
  Synthetic Sequence
attctggctaatacccggagtg

SEQ ID NO: 2 - DNA, 19 bp, Artificial Sequence
  Synthetic Sequence
gcagttcccaggttgagcc

SEQ ID NO: 3 - DNA, 16 bp, Artificial Sequence
  Synthetic Sequence
caggcggtttgctaag

SEQ ID NO: 4 - DNA, 1493 bp, Burkholderia pseudomallei
agtttgatcctggctcagattgaacgctggcggcatgccttacacatgcaagtcgaacggcagcacgggcttcggcctggtggcgagtggcgaacgggtgagtaatacatcggaacatgtcctgtagtgggggatagcccggcgaaagccggattaataccgcatacgatctgaggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgcaagcctgatccagcaatgccgcgtgtgtgaagaaggccttcgggttgtaaagcacttttgtccggaaagaaatcattctggctaatacccggagtggatgacggtaccggaagaataagcaccggctaactacgtgccagcagccgcggtaatacgtagggtgcgagcgttaatcggaattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaagcccgatgagagttgggcgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgta

SEQ ID NO: 5 - DNA, 1528 bp, Burkholderia mallei
gaagagtttgatcctggctcagattgaacgctggcggcatgccttacacatgcaagtcgaacggcagcacgggcttcggcctggtggcgagtggtgaacgggtgagtaatacatcggaacatgtcctgtagtgggggatagcccggcgaaagccggattaataccgcatacgatctgaggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgcaagcctgatccagcaatgccgcgtgtgtgaagaaggccttcgggttgtaaagcacttttgtccggaaagaaatcattctggctaatacccggagtggatgacggtaccggaagaataagcaccggctaactacgtgccagcagccgcggtaatacgtagggtgcgagcgttaatcggaattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaagcccgatgagagttgggcgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtatcggaaggtgcggctggatcacctcctttct

(19) United States
(12) Patent Application Publication
(10) Pub. No.: US 2015/0167056 A1
(43) Pub. Date: Jun. 18, 2015

(54) PRIMERS, ASSAYS AND METHODS FOR DETECTING BURKHOLDERIA PSEUDOMALLEI AND BURKHOLDERIA MALLEI

(71) Applicants: THE TRANSLATIONAL GENOMICS RESEARCH INSTITUTE, PHOENIX, AZ (US); ARIZONA BOARD OF REGENTS ON BEHALF OF NORTHERN ARIZONA UNIVERSITY, Flagstaff, AZ (US)

(72) Inventors: James M. Schupp, Flagstaff, AZ (US); Rebecca E. Colman, Flagstaff, AZ (US); Jordan L. Buchhagen, Flagstaff, AZ (US); Paul S. Keim, Flagstaff, AZ (US); David M. Engelthaler, Flagstaff, AZ (US)

(73) Assignee: THE TRANSLATIONAL GENOMICS RESEARCH INSTITUTE, PHOENIX, AZ (US)

(21) Appl. No.: 14/400,878

(22) PCT Filed: May 15, 2013
(86) PCT No.: PCT/US13/41204

§ 371 Date: Nov. 13, 2014

Related U.S. Application Data
(60) Provisional application No. 61/647,468, filed on May 15, 2012.

(51) Int. Cl. C12Q 1/68

(52) U.S. Cl. CPC C12Q 1/689; C12Q 2600/158

(57) ABSTRACT

Disclosed are methods, assay kits, signature primers, and probes for detecting the presence of Burkholderia pseudomallei and/or Burkholderia mallei in a sample using real-time reverse-transcriptase PCR.

### CROSS-REFERENCE TO RELATED APPLICATIONS

The present application claims the benefit of U.S. Provisional Application No. 61/647,468 filed on May 15, 2012, the content of which is hereby incorporated by reference in its entirety.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 5,025 byte ASCII (text) file named “Seq_List” created on May 15, 2013.

### FIELD OF THE INVENTION

The present invention relates assay kits and methods for detecting the presence of Burkholderia pseudomallei, Burkholderia mallei, or both. Specific aspects of the invention relate to detecting B. pseudomallei and/or B. mallei RNA signatures with real-time Reverse-Transcriptase Polymerase Chain Reaction (RT-PCR).

### BACKGROUND OF THE INVENTION

Melioidosis is an infectious disease endemic to Southeast Asia and northern Australia caused by a Gram-negative bacterium, Burkholderia pseudomallei (White N J, Lancet. 361:1715-1722, 2003). Although melioidosis has historically been considered to be a relatively rare disease it is being diagnosed in an increasing number of countries and with an increasing frequency. This is probably due to a combination of factors, such as recent improvements in diagnostic techniques, a greater awareness of the disease and an increase in global travel from areas of the world where melioidosis is endemic.

Melioidosis can present in a number of forms, which have been described as acute septicaemic, acute pulmonary, sub-acute and chronic diseases. In some cases a persistent sub-clinical infection is established with the subsequent ability to become septicaemic. The factors that influence the outcome of disease are not known, although it has been suggested that differences in the virulence of different strains might contribute to the clinical outcome of disease. In addition, melioidosis is most frequently seen in diabetics, those with impaired cellular immunity or those with a history of drug or alcohol abuse, suggesting that differences in the immunological status of the host might also influence the outcome of the disease.

B. pseudomallei has been isolated, for example, from soil, muddy water and rice paddy fields in the endemic regions. It is estimated that mortality rates within the endemic areas of northeast Thailand and Australia are 50% and 19%, respectively. Burkholderia mallei is very closely related to B. pseudomallei and also causes serious diseases in a host. Delays in detection and treatment of either can be fatal. While long incubation periods have been seen (leading to its nickname of the “Vietnamese time bomb”), typical incubation is from about 24 hours to three weeks. A wide range of symptoms accompanying melioidosis tends to mimic other common bacterial infections. The overall number of melioidosis infections is thought to be greatly underestimated due to lack of reporting, the use of microbiological cultures as the diagnostic standard, and the low number of bacterial cells in common types of diagnostic samples.

Microbiological culture is the conventional method for clinically detecting B. pseudomallei/mallei, for example, from blood, urine, or throat swab samples. B. pseudomallei//mallei antibody detection from serological samples have also been performed with indirect hemagglutination and complement fixation. Polysaccharide microarray and ELISA have also been used. Additionally, real-time PCR has been demonstrated purified samples, for example, by targeting, for example, a DNA sequence from a unique type III secretion system gene.

One of the difficulties with conventional detection methods is the relatively low concentration of B. pseudomallei//mallei as well as its similarity to non-pathogenic bacteria. In a clinical sample, then, it is difficult to achieve high sensitivity and specificity from DNA because of the low copy number.

RNA molecules are transcribed from the deoxyribonucleic acid (DNA) genome of an organism, and lead to the production of proteins. Depending upon the needs of the cell, a single copy DNA gene can be transcribed multiple times in a short period of time. This leads to multiple copies, sometimes thousands, of an RNA transcript being present in a given sample that contains the organism. These RNA molecules can be detected with the use of standard Reverse Transciptase-quantitative Polymerase Chain Reaction (RT-qPCR) assay technology.

Reverse-Transcriptase (RT) real-time PCR has been established as a technology for quantitatively (RT-qPCR) identifying RNA targets. For example, it has been used for detecting RNA biomarkers in certain cancers. Further, due to the RNA genome of many viruses, RT-PCR has been used for detection of certain viruses.

New diagnostic tools utilizing RT-qPCR technology are needed to increase detection of B. pseudomallei in clinical specimens. The use of RT-qPCR allows assays to target a highly expressed RNA transcript instead of a single copy DNA gene in B. pseudomallei. Assays based on amplification of DNA lack the sensitivity required to detect small amounts of B. pseudomallei in clinical samples, which limits their ability to identify infections early on before the infections become life threatening (see, e.g., Tomaso et al., Molecular and Cellular Probes 19: 9-20, 2005).

Because of the clinical importance of B. pseudomallei/mallei and their potential for unpredictable outcomes, fast and accurate detection of the pathogen is vital. The long turnaround of conventional bacterial cultures could mean a difference between life and death. The complexity of the samples, which contain extremely dilute concentrations of the pathogen, and the pathogen's resemblance to other bacteria further complicate its detection. Even so, it is vital that false negatives and false positives be avoided. Therefore, it is desirable to provide a method and assay kit for detecting B. pseudomallei, B. mallei, or both with higher sensitivity than the conventional options, without sacrificing specificity, while also aiming to reduce turnaround time.

### BRIEF SUMMARY OF THE INVENTION

This disclosure demonstrates that RT-qPCR is a useful diagnostic tool to detect the presence of pathogen-specific ribonucleic acid (RNA) molecules in complex samples. Specifically, it demonstrates that B. pseudomallei and/or B. mallei can be detected using RT-PCR by targeting a unique RNA signature on the 16S ribosomal subunit. The systems, methods, and assay kits disclosed herein are shown to be more sensitive than a conventional method that targets DNA in the Type III Secretion system (TTS1) gene, which is also unique to B. pseudomallei.

The present invention provides a method of detecting the presence of B. pseudomallei, B. mallei, or both in a sample. Preferably, it utilizes a forward (Burk16S_Forward: 5′-ATTCTGGCTAATACCCGGAGTG-3′) (SEQ ID NO: 1) and reverse primer (Burk16S_Reverse: 5′-GCAGTTCCCAGGTTGAGCC-3′) (SEQ ID NO: 2) in conjunction with a dual labeled probe oligo (Burk16S_Probe: 5′FAM-CAGGCGGTTTGCTAAG-MGB-3′) (SEQ ID NO: 3) in a reverse-transcriptase polymerase chain reaction (PCR) to detect an RNA product or DNA complement thereof that is specific to B. pseudomallei and/or B. mallei and wherein detection of the RNA product or DNA complement thereof confirms the presence of the B. pseudomallei/mallei in the sample. While the current 16S signature is specific to both B. pseudomallei and B. mallei, other signatures may be used that are specific to only B. pseudomallei or B. mallei.

One or more of the following aspects are present according to embodiments. The detection may be by amplification of cDNA produced in the reverse-transcriptase PCR. The amplification reaction may be quantitative and the quantification may be accomplished with real-time PCR. In the real-time PCR, a dye may be used, selected from the group consisting of: SYBR GREEN, 6-FAM, HEX, JOE, ROX, TET, CY3, CY5, TAMRA, TEXAS RED. One or more bi-labeled probes may be used in the real-time PCR. The quantification may comprise a melt curve analysis. Other dyes from Applied Biosystems, as well as the quencher can be used as well.

In one embodiment, the present invention provides A pair of isolated oligonucleotides for the amplification of a 16S ribosomal RNA nucleic acid from Burkholderia pseudomallei or Burkholderia mallei consisting of: a first oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5; and a second oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5.

In one aspect of the invention, an assay kit is provided for detecting B. pseudomallei, B. mallei, or both, comprising the primers and probe signatures specific to B. pseudomallei and/or B. mallei. In some embodiments, the assay kit further comprises a protocol for identification of the B. pseudomallei, B. mallei, or both. Preferably, it utilizes a forward (Burk16S_Forward: 5′-ATTCTGGCTAATACCCGGAGTG-3′) (SEQ ID NO: 1) and reverse primer (Burk16S_Reverse: 5′-GCAGTTCCCAGGTTGAGCC-3′) (SEQ ID NO: 2) in conjunction with a dual labeled probe oligo (Burk16S_Probe: 5′FAM-CAGGCGGTTTGCTAAG-MGB-3′) (SEQ ID NO: 3) in a reverse-transcriptase polymerase chain reaction (PCR) to detect an RNA product or DNA complement thereof that is specific to B. pseudomallei and/or B. mallei and wherein detection of the RNA product or DNA complement thereof confirms the presence of the B. pseudomallei/mallei in the sample. While the current 16S signature is specific to both B. pseudomallei and B. mallei, other signatures may be used that are specific to only B. pseudomallei or B. mallei.

In another embodiment, the present invention provides a diagnostic kit for detecting the presence of B. pseudomallei, B. mallei, or both in a sample, the kit comprising: a first oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5; a second oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5; and amplification reagents.

In another aspect of the invention, there are provided assay kits for detecting the presence B. pseudomallei, B. mallei, or both. In some embodiments, the kit and assays comprises one or more B. pseudomallei/mallei 16S sequence-specific forward and reverse primers and/or random oligomers (e.g. hexamers) for the reverse transcription, and/or one or more primer pairs for amplification of at least a portion of the cDNA product of the reverse transcription. In a preferred embodiment, the assay kit format is a single step RT-qPCR assay where both primers and the probe are added to the sample RNA and reaction master mix. In some embodiments, the kit comprises one or more probes for detection of the presence of B. pseudomallei, B. mallei, or both. In some embodiments, the kit comprises: a) one or more primer pairs for amplification of a B. pseudomallei and/or B. mallei RNA signature; and/or b) one or more probes for detection of at least one B. pseudomallei and/or B. mallei RNA signature. The probes may be immobilized in a carrier, for example, in the form of microarrays.

Aspects of the disclosed invention includes RNA signatures specific to B. pseudomallei, B. mallei, or both. Other aspects of the disclosed invention also include assays for detecting and using these RNA signatures, for example, to diagnose infections caused by B. pseudomallei, B. mallei, or both, and to guide patient treatment and follow-up, screen at-risk and/or non-symptomatic patients for B. pseudomallei/mallei colonization, detect and quantify B. pseudomallei/mallei from environmental samples. Aspects of the invention further include using such assays individually or in combination with other assays for the aforementioned purposes.

### BRIEF DESCRIPTION OF THE DRAWINGS

FIG. 1 shows amplification plots from validation assays testing the sensitivity and specificity of the Burk-16S assay.

FIG. 2 presents validation results of the Burk-16S assay as traditional real-time PCR.

FIG. 3 shows Reverse Transcriptase quantitative PCR plots of different amounts of B. pseudomallei RNA analyzed with both the Burk 16S assay and the previously described TTS1 assay. The Burk 16S assay consistently amplifies at about 13 CT prior to the TTS1 assay. Note that at 0.01 ng, the TTS1 assay plots are starting to show poor amplification with a 2CT variation among the replicates, whereas the Burk 16S assay plots at 0.01 ng indicate robust amplification and little variation among replicates.

FIG. 4 presents a comparison of Ct values (i.e., the cycle number at which the plot crosses the threshold) for 1 ng of DNA or RNA on the Burk-16S and TTS1 assays as traditional real-time PCR and reverse transcriptase real-time PCR

Elements and facts in the figures are illustrated for simplicity and have not necessarily been rendered according to any particular sequence or embodiment

### DETAILED DESCRIPTION OF THE INVENTION

Aspects and applications of the invention presented herein are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. The full scope of the inventions is not limited to the specific examples that are described below.

“Homologues” of specific genes, primers, and sequences as used herein refers to nucleotide sequences having at least about 40%, including for example at least about any of 50%, 60%, 70%, 80%, 90%, 95%, 98%, 99%, or more sequence identity to the sequence of nucleotide sequences of genes, primers, or probes described herein.

Aspects of the invention include methods for detecting a bacterial species. For example, a pathogenic species, such as B. pseudomallei, B. mallei, or both, may be detected. In some embodiments, the species is detected from a biological sample, such as blood, urine, respiratory secretion, throat swab, saliva, or tissue. According to embodiments, a species-specific signature is selected that is present on the range of >10 times per active cell. In some embodiments, the targeted signature is one or more sequences corresponding to the 16S ribosomal RNA, which is estimated to be present at 10³-10⁴ molecules per actively growing cell. For example, the signature may comprise a sequence of DNA, cDNA, or RNA base pairs that are either homologues or complementary to homologues of one or more sequences of the 16S ribosomal RNA.

According to embodiments RT-PCR is used to increase detection of B. pseudomallei, B. mallei, or both in clinical specimens. The use of RT allows the assay to target a highly expressed RNA transcript instead of a single copy DNA gene.

In some aspects, the present invention is directed to isolated oligonucleotides for the amplification of a 16S ribosomal RNA nucleic acid. As used herein, the term “16S ribosomal RNA nucleic acid” refers to the RNA product of the 16S ribosomal RNA gene and/or its DNA complement. In one embodiment, the 16S ribosomal RNA nucleic acid is the Burkholderia pseudomallei strain K96243 16S ribosomal RNA (NCBI Reference Sequence: NR_{—}074340.1) (SEQ ID NO: 4) shown below:

```
      1 agtttgatcc tggctcagat tgaacgctgg cggcatgcct tacacatgca agtcgaacgg
    61 cagcacgggc ttcggcctgg tggcgagtgg cgaacgggtg agtaatacat cggaacatgt
  121 cctgtagtgg gggatagccc ggcgaaagcc ggattaatac cgcatacgat ctgaggatga
  181 aagcggggga ccttcgggcc tcgcgctata gggttggccg atggctgatt agctagttgg
  241 tggggtaaag gcctaccaag gcgacgatca gtagctggtc tgagaggacg accagccaca
  301 ctgggactga gacacggccc agactcctac gggaggcagc agtggggaat tttggacaat
  361 gggcgcaagc ctgatccagc aatgccgcgt gtgtgaagaa ggccttcggg ttgtaaagca
  421 cttttgtccg gaaagaaatc attctggcta atacccggag tggatgacgg taccggaaga
  481 ataagcaccg gctaactacg tgccagcagc cgcggtaata cgtagggtgc gagcgttaat
  541 cggaattact gggcgtaaag cgtgcgcagg cggtttgcta agaccgatgt gaaatccccg
  601 ggctcaacct gggaactgca ttggtgactg gcaggctaga gtatggcaga ggggggtaga
  661 attccacgtg tagcagtgaa atgcgtagag atgtggagga ataccgatgg cgaaggcagc
  721 cccctgggcc aatactgacg ctcatgcacg aaagcgtggg gagcaaacag gattagatac
  781 cctggtagtc cacgccctaa acgatgtcaa ctagttgttg gggattcatt tccttagtaa
  841 cgtagctaac gcgtgaagtt gaccgcctgg ggagtacggt cgcaagatta aaactcaaag
  901 gaattgacgg ggacccgcac aagcggtgga tgatgtggat taattcgatg caacgcgaaa
  961 aaccttacct acccttgaca tggtcggaag cccgatgaga gttgggcgtg ctcgaaagag
1021 aaccggcgca caggtgctgc atggctgtcg tcagctcgtg tcgtgagatg ttgggttaag
1081 tcccgcaacg agcgcaaccc ttgtccttag ttgctacgca agagcactct aaggagactg
1141 ccggtgacaa accggaggaa ggtggggatg acgtcaagtc ctcatggccc ttatgggtag
1201 ggcttcacac gtcatacaat ggtcggaaca gagggtcgcc aacccgcgag ggggagccaa
1261 tcccagaaaa ccgatcgtag tccggattgc actctgcaac tcgagtgcat gaagctggaa
1321 tcgctagtaa tcgcggatca gcatgccgcg gtgaatacgt tcccgggtct tgtacacacc
1381 gcccgtcaca ccatgggagt gggttttacc agaagtggct agtctaaccg caaggaggac
1441 ggtcaccacg gtaggattca tgactggggt gaagtcgtaa caaggtagcc gta
```

In another embodiment, 16S ribosomal RNA nucleic acid is the Burkholderia mallei ATCC 23344 strain ATCC 23344 16S ribosomal RNA (NCBI Reference Sequence: NR_{—}074299.2) (SEQ ID NO: 5) shown below:

```
      1 gaagagtttg atcctggctc agattgaacg ctggcggcat gccttacaca tgcaagtcga
    61 acggcagcac gggcttcggc ctggtggcga gtggtgaacg ggtgagtaat acatcggaac
  121 atgtcctgta gtgggggata gcccggcgaa agccggatta ataccgcata cgatctgagg
  181 atgaaagcgg gggaccttcg ggcctcgcgc tatagggttg gccgatggct gattagctag
  241 ttggtggggt aaaggcctac caaggcgacg atcagtagct ggtctgagag gacgaccagc
  301 cacactggga ctgagacacg gcccagactc ctacgggagg cagcagtggg gaattttgga
  361 caatgggcgc aagcctgatc cagcaatgcc gcgtgtgtga agaaggcctt cgggttgtaa
  421 agcacttttg tccggaaaga aatcattctg gctaataccc ggagtggatg acggtaccgg
  481 aagaataagc accggctaac tacgtgccag cagccgcggt aatacgtagg gtgcgagcgt
  541 taatcggaat tactgggcgt aaagcgtgcg caggcggttt gctaagaccg atgtgaaatc
  601 cccgggctca acctgggaac tgcattggtg actggcaggc tagagtatgg cagagggggg
  661 tagaattcca cgtgtagcag tgaaatgcgt agagatgtgg aggaataccg atggcgaagg
  721 cagccccctg ggccaatact gacgctcatg cacgaaagcg tggggagcaa acaggattag
  781 ataccctggt agtccacgcc ctaaacgatg tcaactagtt gttggggatt catttcctta
  841 gtaacgtagc taacgcgtga agttgaccgc ctggggagta cggtcgcaag attaaaactc
  901 aaaggaattg acggggaccc gcacaagcgg tggatgatgt ggattaattc gatgcaacgc
  961 gaaaaacctt acctaccctt gacatggtcg gaagcccgat gagagttggg cgtgctcgaa
1021 agagaaccgg cgcacaggtg ctgcatggct gtcgtcagct cgtgtcgtga gatgttgggt
1081 taagtcccgc aacgagcgca acccttgtcc ttagttgcta cgcaagagca ctctaaggag
1141 actgccggtg acaaaccgga ggaaggtggg gatgacgtca agtcctcatg gcccttatgg
1201 gtagggcttc acacgtcata caatggtcgg aacagagggt cgccaacccg cgagggggag
1261 ccaatcccag aaaaccgatc gtagtccgga ttgcactctg caactcgagt gcatgaagct
1321 ggaatcgcta gtaatcgcgg atcagcatgc cgcggtgaat acgttcccgg gtcttgtaca
1381 caccgcccgt cacaccatgg gagtgggttt taccagaagt ggctagtcta accgcaagga
1441 ggacggtcac cacggtagga ttcatgactg gggtgaagtc gtaacaaggt agccgtatcg
1501 gaaggtgcgg ctggatcacc tcctttct
```

In some embodiments, the isolated oligonucleotide is between 10 and 50 nucleotides, e.g., any range between 10 and 50 nucleotides, such as between 10 and 20, between 15 and 25 nucleotides, between 15 and 30 nucleotides, between 18 and 25 nucleotides, between 18 and 30 nucleotides, between 25 and 50 nucleotides, etc.

In some embodiments, the oligonucleotides of the present invention produce positive amplifications with nucleic acid samples from B. pseudomallei and B. mallei with a high degree of sensitivity. As used herein, the term “sensitivity” refers to the proportion or percentage of actual positives that are correctly identified as such. In certain aspects, the oligonucleotides of the present invention produce positive amplifications with nucleic acid samples from B. pseudomallei and B. mallei with at least 50% sensitivity, at least 60% sensitivity, at least 70% sensitivity, at least 80% sensitivity, at least 85% sensitivity, at least 90% sensitivity, at least 95% sensitivity, at least 98% sensitivity, or 100% sensitivity.

In other embodiments, the oligonucleotides of the present invention do not produce positive amplifications with nucleic acid samples from non-B. pseudomallei and non-B. mallei bacterial species with a high degree of specificity. As used herein, the term “specificity” refers to proportion or percentage of negatives which are correctly identified as such. In certain aspects, the oligonucleotides of the present invention do not produce positive amplifications with nucleic acid samples from non-B. pseudomallei and non-B. mallei bacterial species with at least 50% specificity, at least 60% specificity, at least 70% specificity, at least 80% specificity, at least 85% specificity, at least 90% specificity, at least 95% specificity, at least 98% specificity, or 100% specificity.

### Primers and Amplifying Kits

The present invention provides kits comprising primers for amplifying RNA signature products. In some embodiments, RNA signature products are specific to B. pseudomallei, B. mallei, or both. The primers may comprise forward and reverse primers for amplifying the RNA signature products by PCR methods, such as RT-qPCR, which can be monitored in real-time.

The forward and reverse primer of primer pairs described herein for amplification of the RNA signature products are typically 10-50 nucleotides, including for example 12-35 nucleotides, 15-25 nucleotides. In some embodiments, the 5′-end of the forward or reverse primer of the said primer pairs for amplification of the RNA signature products is linked with a universal tagged sequence. The 5′-end of the said universal tagged sequence in some embodiments may be labeled with a fluorescent dye. Exemplary universal tagged sequences are well known in the art.

In some embodiments, the kit comprises at least about two different primer pairs. In some embodiments, the kit comprises at least about three different primer pairs. In some embodiments, the kit comprises at least about any of 3, 4, 5, 6, 7, 8, 9, 10, 20, 30, 40, or 50 different primer pairs. These different primer pairs may amplify one or more RNA signature products. In some embodiments, the kit comprises at least any of 1, 2, 3, 4, 5, 6, 7, 8, 9, 10 pairs of primers listed herein, or homologues thereof.

Suitable amplification reagents that may be used in the kits include enzymes having RNA dependent DNA polymerase activity, enzymes having DNA dependent DNA polymerase activity, enzymes having RNase H activity, and enzymes having RNA polymerase activity.

In some implementations, the disclosed assay kits include reagents available in conventional quantitative PCR assay kits that either utilize a primer/labeled probe combination or primers with SYBR green.

Probes and Assay Kits for Detecting B. Pseudomallei and/or B. Mallei

In some embodiments, the assay kits and assays comprise probes for detecting B. pseudomallei, B. mallei, or both. These probes are capable of hybridizing with the B. pseudomallei and/or B. mallei gene products (including DNA or RNA transcribed from the genes) or amplification of the gene products. In some embodiments, the probes are about 15-50 nucleotides long, including for example about 20-30 nucleotides long. In some embodiments, the probe is dual labeled, with a reporter dye on the 5′ end and a quencher moiety on the 3′ end. In some embodiments, the 5′ end of the probes are linked with an oligonucleotide. For example, the 5′ end of the probes may be linked with an oligo-dT that is about 10-35 nucleotides, including for example about 16-26 nucleotides. In certain embodiments, the primers and probes are designed to be compatible with Taqman® assays and kits.

In certain aspects, the primers are optimized for performance with specific probes. In a non-limiting example, the forward primer and/or reverse primer are optimized for performance with a minor groove binder (MGB) probe. In a specific embodiment, the forward primer (Burk16S_Forward: 5′-ATTCTGGCTAATACCCGGAGTG-3′) (SEQ ID NO: 1) is optimized for performance with an MGB (ABI-Life technologies) Taqman® probe.

In another aspect, the probes are optimized for specific hybridization with a short sequence (e.g., about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, or about 20 nucleotides) of a 16S ribosomal RNA nucleic acid from a Burkholderia pseudomallei and/or a Burkholderia mallei cell. In one embodiment, the dual labeled probe oligo (Burk16S_Probe: 5′FAM-CAGGCGGTTTGCTAAG-MGB-3′) (SEQ ID NO: 3) specifically hybridizes with a 16-nucleotide sequence from a 16S ribosomal RNA nucleic acid from a Burkholderia pseudomallei and/or a Burkholderia mallei cell.

In another aspect, the forward primer and reverse primer are designed to provide for a smaller amplicon, which is more efficient in a Real Time assay format. The smaller amplicon also increases the robustness of the assay when using degraded samples, which is often the case with clinical samples. In certain embodiments, the length of the amplicon is about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 210 nucleotides, about 220 nucleotides, about 230 nucleotides, about 240 nucleotides, about 250 nucleotides, about 260 nucleotides, about 270 nucleotides, about 280 nucleotides, about 290 nucleotides, or about 300 nucleotides. In other embodiments the length of the amplicon is between 100 and 300 nucleotides, e.g., any range between 100 and 300 nucleotides, such as between 100 and 200, between 150 and 250 nucleotides, between 170 and 200 nucleotides, between 180 and 220 nucleotides, between 200 and 300 nucleotides, etc.

In some embodiments, the assay kit comprises a single probe. In some embodiments, the assay kit comprises at least about three different probes. In some embodiments, the assay kit comprises at least about any of 3, 4, 5, 6, 7, 8, 9, 10, 20, 30, 40, or 50 different probes. These probes may detect the same or different B. pseudomallei and/or B. mallei gene products. In some embodiments, the assay kit comprises at least about any of 1, 2, 3, 4, 5, 10, 15, 20, or 21 probes.

The assay kits of the present invention may further comprise other control probes, such as surface chemistry control probe, hybridization control probe, the target of the said hybridization control probe, and negative control probe.

The probes described herein can be immobilized on a carrier, such as a carrier made of silicon, glass slide modified with various functional groups or membranes with various functional groups, preferably glass slide with an aldehyde group.

In some embodiments, the probes described are immobilized in a microarray. “Microarray” and “array,” as used interchangeably herein, comprises a surface with an array, preferably an ordered array, of putative binding (e.g., by hybridization) sites for a biochemical sample (target) which often have undetermined characteristics. In some embodiments, a microarray refers to an assembly of distinct probes immobilized at defined positions on a substrate.

Arrays may be formed on substrates fabricated with materials such as paper, glass, plastic (e.g., polypropylene, nylon, polystyrene), polyacrylamide, nitrocellulose, silicon, optical fiber or any other suitable solid or semisolid support, and configured in a planar (e.g., glass plates, silicon chips) or three dimensional (e.g., pins, fibers, beads, particles, microtiter wells, capillaries) configuration.

Probes forming the array may be attached to the substrate by any number of ways including, but not limiting to, (i) in situ synthesis (e.g., high-density oligonucleotide arrays) using photolithographic techniques; (ii) spotting/printing at medium to low density on glass, nylon or nitrocellulose; (iii) by masking and (iv) by dot-blotting on a nylon or nitrocellulose hybridization membrane, probes may also be non-covalently immobilized on the substrate by hybridization to anchors, by means of magnetic beads, or in a fluid phase such as in microtiter wells or capillaries.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into the amplified nucleic acids. The amplified product is then contacted with a solid substrate, such as a glass slide, which is coated with an aldehyde or another reactive group which will form a covalent link with the reactive group that is on the amplified product and become covalently attached to the glass slide. Microarrays comprising the amplified products can be fabricated using a Biodot (BioDot, Inc. Irvine, Calif.) spotting apparatus and aldehyde-coated glass slides (CEL Associates, Houston, Tex.). Amplification products can be spotted onto the aldehyde-coated slides, and processed according to published procedures (Schena et al., Proc. Natl. Acad. Sci. U.S.A. (1995) 93:10614-10619). Arrays can also be printed by robotics onto glass, nylon (Ramsay, G., Nature Biotechnol. (1998), 16:40-44), polypropylene (Matson, et al., Anal Biochem. (1995), 224(1): 110-6), and silicone slides (Marshall, A. and Hodgson, J., Nature Biotechnol. (1998), 16:27-31). Other approaches to array assembly include fine micropipetting within electric fields (Marshall and Hodgson, supra), and spotting the polynucleotides directly onto positively coated plates. Methods such as those using amino propyl silicon surface chemistry are also known in the art.

Typically, the assay kits use an amplification assay, wherein the signal is amplified and detected during the PCR.

The assay kits of the present invention may also include the reaction solutions for performing PCR and hybridization, and 50% dimethyl sulphoxide (DMSO) as the blank control of the hybridization reaction.

In certain embodiments, the assay kit further comprises instructions for using the assay kit for detecting B. pseudomallei, B. mallei, or both. For example, the assay kit may comprise instruction on performing real-time RT-PCR reactions, and interpretations of real-time RT-PCR results, and/or instructions for carrying out methods described herein. In some embodiments, the assay kit may further comprise reagents for hybridization reactions and interpretation of hybridization results.

In some embodiments, the kit or assay further comprises software for analyzing experimental results using kits, assays or microarrays described herein.

Kits according to the invention include one or more reagents useful for practicing one or more assay methods of the invention. A kit generally includes a package with one or more containers holding the reagent(s) (e.g., primers and/or probe(s)), as one or more separate compositions or, optionally, as admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits according to the invention generally include instructions for carrying out one or more of the methods of the invention. Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

Methods of Detecting B. pseudomallei and B. mallei

Also provided are methods for detection of the presence of B. pseudomallei and/or B. mallei bacteria using the aforementioned assay kits for detection.

In some embodiments, there is provided a method for detecting a B. pseudomallei and/or B. mallei bacteria, the method comprising: a) performing RT-PCR using at least one sequence-specific B. pseudomallei and/or B. mallei 16S forward and reverse primer pair and/or one or more oligo (dT) or random hexamer primers in the presence of reverse transcriptase; and b) amplifying the cDNA product of the reverse transcriptase reaction with real-time PCR using at least one pair of primers specific to B. pseudomallei and/or B. mallei bacteria 16S cDNA.

The concentrations of the forward and reverse primer of the PCR primer pairs can be equal or non-equal. For example, in some embodiments, one of the primers is tagged with a universal tagged sequence at its 5′ end, and the concentration of the primer whose 5′ end is linked with the said 5′-universal tagged sequence is 5-100 folds to the concentration of another primer. In some embodiments, the concentration of the tagged sequence is about 2.5 folds higher than that of the untagged sequence.

In some embodiments, the temperature cycles of the said PCR amplification includes two steps: the cycles in the first step are composed of denaturation, annealing and extension, comprising 10-30 cycles; the cycles in the second step are composed of denaturation and extension, including 10-30 cycles. In some embodiments, the denaturation temperature is 94° C., the annealing temperature is 50-70° C., preferably 55° C., and the extension temperature in the second step is 60-80° C., preferably is 70° C.

In some embodiments, the PCR comprises a reverse-transcriptase (RT) PCR. By way of example, reagents for RT-PCR may comprise one or more of: RNase-free water, template RNA, reverse transcription buffer, reverse transcriptase (e.g. rTth DNA Polymerase, MULTISCRIBE reverse transcriptase, etc.), MnCl₂ and/or Mg Cl₂, dNTPs, one or more sequence-specific primers, and/or random oligomers such as hexamers. In some embodiments, on or more dNTP is a labeled dNTP, such as CY5- or CY3-labeled dCTP. In some embodiments, the RNA is heated in the presence of the primer(s) at about 70° C. to denature the RNA secondary structure, after which the solution is rapidly cooled to allow annealing. The reverse transcription reaction is extended at 35-50° C., preferably 42° C. In some implementations, the reaction is again heated to about 70° C. to denature the reverse transcriptase. In some implementations, RNase is added to remove the RNA template. In other embodiments, the reverse transcription is carried out by heating the solution to about 60-80° C., preferably 70° C. on a pre-heated plate, for about 15 minutes, for example, and cooling the solution back to room temperature. It is understood that, in some applications, other protocols may be used, depending on, for example, which type of reverse transcriptase and which primers are used.

In some implementations, the reverse-transcriptase reaction solution is prepared together with the solutions for real-time PCR amplification, consisting of a single reverse transcription step of 45° C. for 10 minutes, then a reverse transcriptase deactivation/initial denaturation step of 95° C. for 10 minutes followed by 40 cycles of a two step amplification (95° C. for 15 seconds followed by 62° C. for 45 seconds)

In some implementations, the reverse transcription and amplification are performed in separate steps with different solutions. For example, in some implementations, after the reverse transcription, additional reagents, such as chelating buffer, MgCl₂, and forward and reverse primers and, in some cases, probes, may be added for the real-time PCR.

In some embodiments, the PCR is a multiplex asymmetric PCR. In the multiplex asymmetric PCR of the present invention, DNA polymerase, dNTP, Mg²⁺ concentration and the compounds of the buffer are same as that in traditional PCR, and they can be optimized according to different reactions. The difference lies in the primers: one gene-specific primer is same as that in traditional PCR, while another gene-specific primer is added an oligonucleotide which is unrelated to the target sequence. The concentrations of these two primers can be equal. The different gene-specific primers can be added the same tagged sequence. The temperature cycles of one exemplary multiplex PCR include two steps: the first step is same as the traditional PCR, including denaturation, annealing and extension. The annealing temperature is adjusted according to Tm of the gene-specific primer; similarly, the extension time can be adjusted according to the length of the amplified fragment. After about 20 cycles, the reaction begins to perform the second step. The temperature cycles of the second step only include denaturation and extension, and the temperature of extension is about 70° C. In the first 20 cycles of amplification reaction, the primer pairs can perform the common PCR due to the annealing temperature is equivalent to Tm of the gene-specific primers. While in the latter 20 cycles of amplification, only the tagged gene-specific primer can anneal to the target, so that the single-stranded products are produced. The primers included in the kit for detection of B. pseudomallei/mallei are those disclosed above.

In order to detect B. pseudomallei/mallei, the signals of the targets and the probes are detected and analyzed by, such as, typically the fluorescence scanner, and then analyzed the hybridization signals by an appropriate software.

Nucleic acids, including oligonucleotide probes, in the methods and compositions described herein may be labeled with a reporter. A reporter is a molecule that facilitates the detection of a molecule to which it is attached. Numerous reporter molecules that may be used to label nucleic acids are known. Direct reporter molecules include fluorophores, chromophores, and radiophores. Non-limiting examples of fluorophores include, a red fluorescent squarine dye such as 2,4-Bis[1,3,3-trimethyl-2-indolinylidenemethyl]cyclobutenediylium-1,3-dioxolate, an infrared dye such as:

2,4Bis[3,3-dimethyl-2-(1H-benz[e]indolinylidenemethyl)]cyclobutenediylium-1,3-dioxolate, or an orange fluorescent squarine dye such as 2,4-Bis[3,5-dimethyl-2-pyrrolyl]cyclobutenediylium-1,3-diololate. Additional non-limiting examples of fluorophores include quantum dots, Alexa Fluor® dyes, AMCA, BODIPY® 630/650, BODIPY® 650/665, BODIPY®-FL, BODIPY®-R6G, BODIPY®-TMR, BODIPY®-TRX, Cascade Blue®, CyDye™, including but not limited to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, or Texas Red®. A signal amplification reagent, such as tyramide (PerkinElmer), may be used to enhance the fluorescence signal. Indirect reporter molecules include biotin, which must be bound to another molecule such as streptavidin-phycoerythrin for detection. In a multiplex reaction, the reporter attached to the primer or the dNTP may be the same for all reactions in the multiplex reaction if the identities of the amplification products can be determined based on the specific location or identity of the solid support to which they hybridize.

It is also contemplated that fluorophore/quencher-based detection systems may be used with the methods and compositions disclosed herein. When a quencher and fluorophore are in proximity to each other, the quencher quenches the signal produced by the fluorophore. A conformational change in the nucleic acid molecule separates the fluorophore and quencher to allow the fluorophore to emit a fluorescent signal. Fluorophore/quencher-based detection systems reduce background and therefore allow for higher multiplexing of primer sets compared to free floating fluorophore methods, particularly in closed tube and real-time detection systems.

In particular embodiments, molecules useful as quenchers include, but are not limited to tetramethylrhodamine (TAMRA), DABCYL (DABSYL, DABMI or methyl red) anthroquinone, nitrothiazole, nitroimidazole, malachite green, Black Hole Quenchers®, e.g., BHQ1 (Biosearch Technologies), Iowa Black® or ZEN quenchers (from Integrated DNA Technologies, Inc.) (e.g., 3′ Iowa Black® RQ-Sp aka 3IABRQSp and 3′ Iowa Black® FQ aka 3IABkFQ), TIDE Quencher 2 (TQ2) and TIDE Quencher 3 (TQ3) (from AAT Bioquest).

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Preferably, commercially available linking moieties are employed that can be attached to an oligonucleotide during synthesis, e.g., available from Integrated DNA Technologies (Coralville, Iowa) or Eurofins MWG Operon (Huntsville, Ala.).

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

The amplifying step can be performed using any type of nucleic acid template-based method, such as PCR technology.

The polymerase chain reaction (PCR) is a technique widely used in molecular biology to amplify a piece of DNA by in vitro enzymatic replication. Typically, PCR applications employ a heat-stable DNA polymerase, such as Taq polymerase. This DNA polymerase enzymatically assembles a new DNA strand from nucleotides (dNTPs) using single-stranded DNA as template and DNA primers to initiate DNA synthesis. A basic PCR reaction requires several components and reagents including: a DNA template that contains the target sequence to be amplified; one or more primers, which are complementary to the DNA regions at the 5′ and 3′ ends of the target sequence; a DNA polymerase (e.g., Taq polymerase) that preferably has a temperature optimum at around 70° C.; deoxynucleotide triphosphates (dNTPs); a buffer solution providing a suitable chemical environment for optimum activity and stability of the DNA polymerase; divalent cations, typically magnesium ions (Mg2+); and monovalent cation potassium ions.

PCR technology relies on thermal strand separation followed by thermal dissociation. During this process, at least one primer per strand, cycling equipment, high reaction temperatures and specific thermostable enzymes are used (U.S. Pat. Nos. 4,683,195 and 4,883,202). Alternatively, it is possible to amplify the DNA at a constant temperature (Nucleic Acids Sequence Based Amplification (NASBA) Kievits, T., et al., J. Virol Methods, 1991; 35, 273-286; and Malek, L. T., U.S. Pat. No. 5,130,238; T7 RNA polymerase-mediated amplification (TMA) (Giachetti C, et al. J Clin Microbiol 2002 July; 40(7):2408-19; or Strand Displacement Amplification (SDA), Walker, G. T. and Schram, J. L., European Patent Application Publication No. 0 500 224 A2; Walker, G. T., et al., Nuc. Acids Res., 1992; 20, 1691-1696).

Thermal cycling subjects the PCR sample to a defined series of temperature steps. Each cycle typically has 2 or 3 discrete temperature steps. The cycling is often preceded by a single temperature step (“initiation”) at a high temperature (>90° C.), and followed by one or two temperature steps at the end for final product extension (“final extension”) or brief storage (“final hold”). The temperatures used and the length of time they are applied in each cycle depend on a variety of parameters. These include the enzyme used for DNA synthesis, the concentration of divalent ions and dNTPs in the reaction, and the melting temperature (Tm) of the primers. Commonly used temperatures for the various steps in PCR methods are: initialization step—94-96° C.; denaturation step—94-98° C.; annealing step—50-65° C.; extension/elongation step—70-74° C.; final elongation—70-74° C.; final hold—4-10° C.

Real-time polymerase chain reaction, also called quantitative real time polymerase chain reaction (QRT-PCR) or kinetic polymerase chain reaction, is used to amplify and simultaneously quantify a targeted DNA molecule. It enables both detection and quantification (as absolute number of copies or relative amount when normalized to DNA input or additional normalizing genes) of a specific sequence in a DNA sample. Real-time PCR may be combined with reverse transcription polymerase chain reaction to quantify low abundance RNAs. Relative concentrations of DNA present during the exponential phase of real-time PCR are determined by plotting fluorescence against cycle number on a logarithmic scale. Amounts of DNA may then be determined by comparing the results to a standard curve produced by real-time PCR of serial dilutions of a known amount of DNA.

Multiplex-PCR and multiplex real-time PCR use of multiple, unique primer sets within a single PCR reaction to produce amplicons of different DNA sequences. By targeting multiple genes at once, additional information may be gained from a single test run that otherwise would require several times the reagents and more time to perform Annealing temperatures for each of the primer sets should be optimized to work within a single reaction.

Mulitplex-PCR and multiplex real-time PCR may also use unique sets or pools of oligonucleotide probes to detect multiple amplicons at once. In some embodiments, the method of the present invention comprises multiplex quantitative real time PCR (qPCR) with unique pools of oligonucleotide probes.

The methods disclosed herein may also utilize asymmetric priming techniques during the PCR process, which may enhance the binding of the reporter probes to complimentary target sequences. Asymmetric PCR is carried with an excess of the primer for the chosen strand to preferentially amplify one strand of the DNA template more than the other.

Amplified nucleic acid can be detected using a variety of detection technologies well known in the art. For example, amplification products may be detected using agarose gel by performing electrophoresis with visualization by ethidium bromide staining and exposure to ultraviolet (UV) light, by sequence analysis of the amplification product for confirmation, or hybridization with an oligonucleotide probe.

The oligonucleotide probe may comprise a flourophore and/or a quencher. The oligonucleotide probe may also contain a detectable label including any molecule or moiety having a property or characteristic that is capable of detection, such as, for example, radioisotopes, fluorophores, chemiluminophores, enzymes, colloidal particles, and fluorescent microparticles.

Probe sequences can be employed using a variety of methodologies to detect amplification products. Generally all such methods employ a step where the probe hybridizes to a strand of an amplification product to form an amplification product/probe hybrid. The hybrid can then be detected using labels on the primer, probe or both the primer and probe. Examples of homogeneous detection platforms for detecting amplification products include the use of FRET (fluorescence resonance energy transfer) labels attached to probes that emit a signal in the presence of the target sequence. “TaqMan®” assays described in U.S. Pat. Ser. Nos. 5,210,015; 5,804,375; 5,487,792 and 6,214,979 (each of which is herein incorporated by reference) and Molecular Beacon assays described in U.S. Pat. No. 5,925,517 (herein incorporated by reference) are examples of techniques that can be employed to detect nucleic acid sequences. With the “TaqMan®” assay format, products of the amplification reaction can be detected as they are formed or in a so-called “real time” manner. As a result, amplification product/probe hybrids are formed and detected while the reaction mixture is under amplification conditions.

For example, the PCR probes may be TaqMan® probes that are labeled at the 5′ end with a fluorophore and at the 3′-end with a quencher molecule. Suitable fluorophores and quenchers for use with TaqMan® probes are disclosed in U.S. Pat. Nos. 5,210,015, 5,804,375, 5,487,792 and 6,214,979 and WO 01/86001 (Biosearch Technologies). Quenchers may be Black Hole Quenchers disclosed in WO 01/86001.

Nucleic acid hybridization can be done using techniques and conditions known in the art. Specific hybridization conditions will depend on the type of assay in which hybridization is used. Hybridization techniques and conditions can be found, for example, in Tijssen (1993) Laboratory Techniques in Biochemistry and Molecular Biology—Hybridization with Nucleic Acid Probes, Part 1, Chapter 2 (Elsevier, N.Y.); and Ausubel et al., eds. (1995) Current Protocols in Molecular Biology, Chapter 2 (Greene Publishing and Wiley-Interscience, New York) and Sambrook et al. (1989) Molecular Cloning. A Laboratory Manual (2d ed., Cold Spring Harbor Laboratory Press, Plainview, N.Y.).

Hybridization of nucleic acid may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified. Alternatively, stringency conditions can be adjusted to allow some mismatching in sequences so that lower degrees of similarity are detected.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1×to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours, or less depending on the assay format.

It should be noted that the oligonucleotides of this disclosure can be used as primers or probes, depending on the intended use or assay format. For example, an oligonucleotide used as a primer in one assay can be used as a probe in another assay. The grouping of the oligonucleotides into primer pairs and primer/probe sets reflects certain implementations only. However, the use of other primer pairs comprised of forward and reverse primers selected from different preferred primer pairs is specifically contemplated.

Quantitative Real-Time PCR (qPCR) Detection Chemistries

There are several commercially available nucleic acid detection chemistries currently used in qPCR. These chemistries include DNA binding agents, FRET based nucleic acid detection, hybridization probes, molecular beacons, hydrolysis probes, and dye-primer based systems. Each of these chemistries is discussed in more detail below.

### DNA Binding Agents

The first analysis of kinetic PCR was performed by Higuchi et al. who used ethidium bromide to bind double-stranded DNA products (Higuchi et al., Biotechnol., 10: 412-417, 1992; Higuchi et al., Biotechnol., 11:1026-1030, 1993; U.S. Pat. No. 5,994,056; U.S. Published Application No. 2001/6171785). Ethidium bromide, like all other DNA binding agents used in kinetic PCR, is able to increase in fluorescent intensity upon binding. The resulting increase in signal can be recorded over the course of the reaction, and plotted versus the cycle number. Recording the data in this way is more indicative of the initial concentration of the sample of interest compared to end-point analysis.

Binding dyes are relatively inexpensive as compared to other detection chemistries. The advantages of using these binding dyes are their low cost and excellent signal to noise ratios. Disadvantages include their non-specific binding properties to any double-stranded DNA in the PCR reaction, including amplicons created by primer-dimer formations (Wittwer et al., Biotechniques, 22:130-138, 1997). In order to confirm the production of a specific amplicon, a melting curve analysis should be performed (Ishiguro et al., Anal. Biochemistry, 229(2): 207-213, 1995). Another drawback is that amplification of a longer product will generate more signal than a shorter one. If amplification efficiencies are different, quantification may be even more inaccurate (Bustin et al., J. Biomol. Tech., 15:155-166, 2004).

SYBR® Green I from Invitrogen™ (Carlsbad, Calif.) is a popular intercalating dye (Bengtsson et al., Nucleic Acids Res., 31:e45, 2003). SYBR® Green I is a cyclically substituted asymmetric cyanine dye (Zipper et al., Nucleic Acids Res., 32(12):103, 2004; U.S. Pat. No. 5,436,134; U.S. Pat. No. 5,658,751). A minor groove binding asymmetric cyanine dye known as BEBO, has been used in real-time PCR. BEBO causes a non-specific increase in fluorescence with time, perhaps due to a slow aggregation process and is less sensitive compared to SYBR® Green I. A similar dye called BOXTO has also been reported for use in qPCR (Bengtsson et al., Nucleic Acids Res., 31:e45, 2003; U.S. Published Application No. 2006/0211028). Like BEBO, BOXTO is less sensitive than SYBR® Green I (U.S. Published Application No. 2006/0211028).

Other common reporters include YO-PRO-1 and thiazole orange (TO) which are intercalating asymmetric cyanine dyes (Nygren et al., Biopolymers, 46:39-51, 1998). While these dyes exhibit large increases in fluorescence intensity upon binding, TO and Oxazole Yellow (YO) have been reported to perform poorly in real-time PCR (Bengtsson et al., Nucleic Acids Res., 31:e45, 2003). Other dyes that may be used include, but are not limited to, pico green, acridinium orange, and chromomycin A3 (U.S. Published Application No. 2003/6569627). Dyes that may be compatible with real-time PCR can be obtained from various vendors such as, Invitrogen, Cambrex Bio Science (Walkersville, Md.), Rockland Inc. (Rockland, Me.), Aldrich Chemical Co. (Milwaukee, Wis.), Biotium (Hayward, Calif.), TATAA Biocenter AB. (Goteborg, Sweden) and Idaho Technology (Salt Lake City, Utah) (U.S. Published Application No. 2007/0020672).

A dye known as EvaGreen™ (Biotium) has shown promise in that it is designed to not inhibit PCR, and is more stable in alkaline conditions as compared to SYBR® Green I (Dorak, In: Real-time PCR, Bios Advanced Methods, 1st Ed., Taylor & Francis, 2006; U.S. Published Application No. 2006/0211028). Other newer dyes include the LCGreen® dye family (Idaho Technology). LCGreen® I and LCGreen® Plus are the most commercially competitive of these dyes. LCGreen® Plus is considerably brighter than LCGreen® (U.S. Published Application No. 2007/0020672; Dorak, In: Real-time PCR, Bios Advanced Methods, 1st Ed., Taylor & Francis, 2006; U.S. Published Application No. 2005/0233335; U.S. Published Application No. 2066/0019253).

### FRET Based Nucleic Acid Detection

Many real-time nucleic acid detection methods utilize labels that interact by Förster Resonance Energy Transfer (FRET). This mechanism involves a donor and acceptor pair wherein the donor molecule is excited at a particular wavelength, and subsequently transfers its energy non-radiatively to the acceptor molecule. This typically results in a signal change that is indicative of the proximity of the donor and acceptor molecules to one another.

Early methods of FRET based nucleic acid detection that lay a foundation for this technology in general, include work by Heller et al. (U.S. Pat. Nos. 4,996,143; 5,532,129; and 5,565,322, which are incorporated by reference). These patents introduce FRET based nucleic acid detection by including two labeled probes that hybridize to the target sequence in close proximity to each other. This hybridization event causes a transfer of energy to produce a measurable change in spectral response, which indirectly signals the presence of the target.

Cardullo et al. (incorporated by reference) established that fluorescence modulation and nonradiative fluorescence resonance energy transfer can detect nucleic acid hybridization in solution (Cardullo et al., Proc. Natl. Acad. Sci. USA, 85:8790-8804, 1988). This study used three FRET based nucleic acid detection strategies. The first includes two 5′ labeled probes that were complementary to one another, allowing transfer to occur between a donor and acceptor fluorophore over the length of the hybridized complex. In the second method, fluorescent molecules were covalently attached to two nucleic acids, one at the 3′ end and the other at the 5′ end. The fluorophore-labeled nucleic acids hybridized to distinct but closely spaced sequences of a longer, unlabeled nucleic acid. Finally, an intercalating dye was used as a donor for an acceptor fluorophore that was covalently attached at the 5′ end of the probe.

Morrison et al. (Morrison et al., Anal. Biochem., 183:231-244, 1989), incorporated by reference, used complementary labeled probes to detect unlabeled target DNA by competitive hybridization, producing fluorescence signals which increased with increasing target DNA concentration. In this instance, two probes were used that were complementary to one another and labeled at their 5′ and 3′ ends with fluorescein and fluorescein quencher, respectively. Later work also showed that fluorescence melting curves could be used to monitor hybridization (Morrison et al., Biochemistry, 32:3095-3104, 1993).

### Hybridization Probes

Hybridization probes used in real-time PCR were developed mainly for use with the Roche LightCycler® instruments (U.S. Published Application No. 2001/6174670; U.S. Published Application No. 2000/6140054). These are sometimes referred to as FRET probes, LightCycler® probes, or dual FRET probes (Espy et al., Clin. Microbiol. Rev., 19(1):165-256, 2006).

Hybridization probes are used in a format in which FRET is measured directly (Wilhelm and Pingoud, Chem. BioChem., 4:1120-1128, 2003). Each of the two probes is labeled with a respective member of a fluorescent energy transfer pair, such that upon hybridization to adjacent regions of the target DNA sequence, the excitation energy is transferred from the donor to the acceptor, and subsequent emission by the acceptor can be recorded as reporter signal (Wittwer et al., Biotechniques, 22:130-138, 1997). The two probes anneal to the target sequence so that the upstream probe is fluorescently labeled at its 3′ end and the downstream probe is labeled at its 5′ end. The 3′ end of the downstream probe is typically blocked by phosphorylation or some other means to prevent extension of the probe during PCR. The dye coupled to the 3′ end of the upstream probe is sufficient to prevent extension of this probe. This reporter system is different from other FRET based detection methods (molecular beacons, TaqMan®, etc.) in that it uses FRET to generate rather than to quench the fluorescent signal (Dorak, In: Real-time PCR, Bios Advanced Methods, 1st Ed., Taylor & Francis, 2006).

Typical acceptor fluorophores include the cyanine dyes (Cy3 and Cy5), 6-carboxy-4,7,2′,7′-tetrachlorofluorescein (TET), 6-carboxy-N,N,N′,N′-tetramethylrhodamine (TAMRA), and 6-carboxyrhodamine X (ROX). Donor fluorophores are usually 6-carboxyfluoroscein (FAM) (Wilhelm and Pingoud, Chem. BioChem., 4:1120-1128, 2003). Hybridization probes are particularly advantageous for genotyping and mismatch detection. Melting curve analysis can be performed in addition to the per-cycle monitoring of fluorescence during the PCR reaction. A slow heating of the sample after probe hybridization can provide additional qualitative information about the sequence of interest (Lay and Wittwer, Clin. Chem., 1997; 43: 2262-2267, 1997; Bernard et al., Am. J. Pathol., 153:1055-1061, 1998; Bernard et al., Anal. Biochem., 255:101-107, 1998). Base-pair mismatches will shift the stability of a duplex, in varying amounts, depending on the mismatch type and location in the sequence (Guo et al., Nat. Biotechnol., 4:331-335, 1997).

### Molecular Beacons

Molecular beacons, also known as hairpin probes, are stem-loop structures that open and hybridize in the presence of a complementary target sequence, typically causing an increase in fluorescence (U.S. Pat. No. 5,925,517); U.S. Published Application No. 2006/103476). Molecular beacons typically have a nucleic acid target complement sequence flanked by members of an affinity pair that, under assay conditions in the absence of target, interact with one another to form a stem duplex. Hybridization of the probes to their preselected target sequences produces a conformational change in the probes, forcing the “arms” apart and eliminating the stem duplex and thereby separating the fluorophore and quencher.

### Hydrolysis Probes

Hydrolysis probes, also known as the TaqMan® assay (U.S. Pat. No. 5,210,015), are popular because they only involve a single probe per target sequence, as opposed to two probes (as in hybridization probes). This results in a cost savings per sample. The design of these probes is also less complicated than that of molecular beacons. These are typically labeled with a reporter on the 5′ end and a quencher on the 3′ end. When the reporter and quencher are fixed onto the same probe, they are forced to remain in close proximity. This proximity effectively quenches the reporter signal, even when the probe is hybridized to the target sequence. During the extension or elongation phase of the PCR reaction, a polymerase known as Taq polymerase is used because of its 5′ exonuclease activity. The polymerase uses the upstream primer as a binding site and then extends. Hydrolysis probes are cleaved during polymerase extension at their 5′ end by the 5′-exonuclease activity of Taq. When this occurs, the reporter fluorophore is released from the probe, and subsequently, is no longer in close proximity to the quencher. This produces a perpetual increase in reporter signal with each extension phase as the PCR reaction continues cycling. In order to ensure maximal signal with each cycle, hydrolysis probes are designed with a Tm that is roughly 10° C. higher than the primers in the reaction.

However, the process of cleaving the 5′ end of the probe need not require amplification or extension of the target sequence (U.S. Pat. No. 5,487,972). This is accomplished by placing the probe adjacent to the upstream primer, on the target sequence. In this manner, sequential rounds of annealing and subsequent probe hydrolysis can occur, resulting in a significant amount of signal generation in the absence of polymerization. Uses of the real-time hydrolysis probe reaction are also described in U.S. Pat. Nos. 5,538,848 and 7,205,105, both of which are incorporated by references.

### Dye-Primer Based Systems

There are numerous dye-labeled primer based systems available for real-time PCR. These range in complexity from simple hairpin primer systems to more complex primer structures where the stem-loop portion of the hairpin probe is attached via a non-amplifiable linker to the specific PCR primer. These methods have the advantage that they do not require an additional intervening labeled probe that is essential for probe-based assay systems and they also allow for multiplexing that is not possible with DNA binding dyes. However, the success of each of these methods is dependent upon careful design of the primer sequences.

Hairpin primers contain inverted repeat sequences that are separated by a sequence that is complementary to the target DNA (Nazarenko et al., Nucleic Acids Res., 25(12):2516-2521, 1997; Nazarenko et al., Nucleic Acids Res., 30(9):37, 2002; U.S. Pat. No. 5,866,336). The repeats anneal to form a hairpin structure, such that a fluorophore at the 5′-end is in close proximity to a quencher at the 3′-end, quenching the fluorescent signal. The hairpin primer is designed so that it will preferentially bind to the target DNA, rather than retain the hairpin structure. As the PCR reaction progresses, the primer anneals to the accumulating PCR product, the fluorophore and quencher become physically separated, and the level of fluorescence increases.

Invitrogen's LUX™ (Light Upon eXtension) primers are fluorogenic hairpin primers which contain a short 4-6 nucleotide extension at the 5′ end of the primer that is complementary to an internal sequence near the 3′ end and overlaps the position of a fluorophore attached near the 3′ end (Chen et al., J. Virol. Methods, 122(1):57-61, 2004; Bustin, J. Mol. Endocrinol., 29(1):23-39, 2002). Basepairing between the complementary sequences forms a double-stranded stem which quenches the reporter dye that is in close proximity at the 3′ end of the primer. During PCR, the LUX™ primer is incorporated into the new DNA strand and then becomes linearized when a new complementary second strand is generated. This structural change results in an up to 10-fold increase in the fluorescent signal. These primers can be difficult to design and secondary structure must be carefully analyzed to ensure that the probe anneals preferentially to the PCR product. Design and validation services for custom LUX™ primers are available from Invitrogen.

More recently, hairpin probes have become part of the PCR primer (Bustin, J. Mol. Endocrinol., 29(1):23-39, 2002). In this approach, once the primer is extended, the sequence within the hairpin anneals to the newly synthesized PCR product, disrupting the hairpin and separating the fluorophore and quencher.

Scorpion® primers are bifunctional molecules in which an upstream hairpin probe sequence is covalently linked to a downstream primer sequence (U.S. Published Application No. 2001/6270967; U.S. Published Application No. 2005/0164219; Whitcombe et al., Nat. Biotechnol., 17:804-807, 1999). The probe contains a fluorophore at the 5′ end and a quencher at the 3′ end. In the absence of the target, the probe forms a 6-7 base stem, bringing the fluorophore and quencher in close proximity and allowing the quencher to absorb the fluorescence emitted by the fluorophore. The loop portion of the scorpion probe section consists of sequence complementary to a portion of the target sequence within 11 bases downstream from the 3′ end of the primer sequence. In the presence of the target, the probe becomes attached to the target region synthesized in the first PCR cycle. Following the second cycle of denaturation and annealing, the probe and the target hybridize. Denaturation of the hairpin loop requires less energy than the new DNA duplex produced. Thus, the scorpion probe loop sequence hybridizes to a portion of the newly produced PCR product, resulting in separation of the fluorophore from the quencher and an increase in the fluorescence emitted.

As with all dye-primer based methods, the design of Scorpion primers follows strict design considerations for secondary structure and primer sequence to ensure that a secondary reaction will not compete with the correct probing event. The primer pair should be designed to give an amplicon of approximately 100-200 bp. Ideally, the primers should have as little secondary structure as possible and should be tested for hairpin formation and secondary structures. The primer should be designed such that it will not hybridize to the probe element as this would lead to linearization and an increase in non-specific fluorescence emission. The Tm's of the two primers should be similar and the stem Tm should be 5-10° C. higher than the probe Tm. The probe sequence should be 17-27 bases in length and the probe target should be 11 bases or less from the 3′ end of the scorpion. The stem sequence should be 6 to 7 bases in length and should contain primarily cytosine and guanine The 5′ stem sequence should begin with a cytosine as guanine may quench the fluorophore. Several oligonucleotide design software packages contain algorithms for Scorpion primer design and custom design services are available from some oligonucleotide vendors as well.

The Plexor™ system from Promega is a real-time PCR technology that has the advantage that there are no probes to design and only one PCR primer is labeled (U.S. Pat. No. 5,432,272; U.S. Published Application No. 2000/6140496; U.S. Published Application No. 2003/6617106). This technology takes advantage of the specific interaction between two modified nucleotides, isoguanine (iso-dG) and 5′-methylisocytosine (iso-dC) (Sherrill et al., J. Am. Chem. Soc., 126:4550-4556, 2004; Johnson et al., Nucl. Acids Res., 32:1937-1941, 2004; Moser et al., Nucl. Acids Res., 31:5048-5053, 2003). Main features of this technology are that the iso-bases will only base pair with the complementary iso-base and DNA polymerase will only incorporate an iso-base when the corresponding complementary iso-base is present in the existing sequence. One PCR primer is synthesized with a fluorescently-labeled iso-dC residue as the 5′-terminal nucleotide. As amplification progresses, the labeled primer is annealed and extended, becoming incorporated in the PCR product. A quencher-labeled iso-dGTP (dabsyl-iso-dGTP), available as the free nucleotide in the PCR master mix, specifically base pairs with the iso-dC and becomes incorporated in the complementary PCR strand, quenching the fluorescent signal. Primer design for the Plexor system is relatively simple as compared to some of the other dye-primer systems and usually follows typical target-specific primer design considerations. A web-based Plexor Primer Design Software, available from Promega, assists in selecting the appropriate dye and quencher combinations, and provides links to oligonucleotide suppliers licensed to provide iso-base containing primers.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

### EXAMPLES

### Example 1

### Validation of the Burk-16S Assay

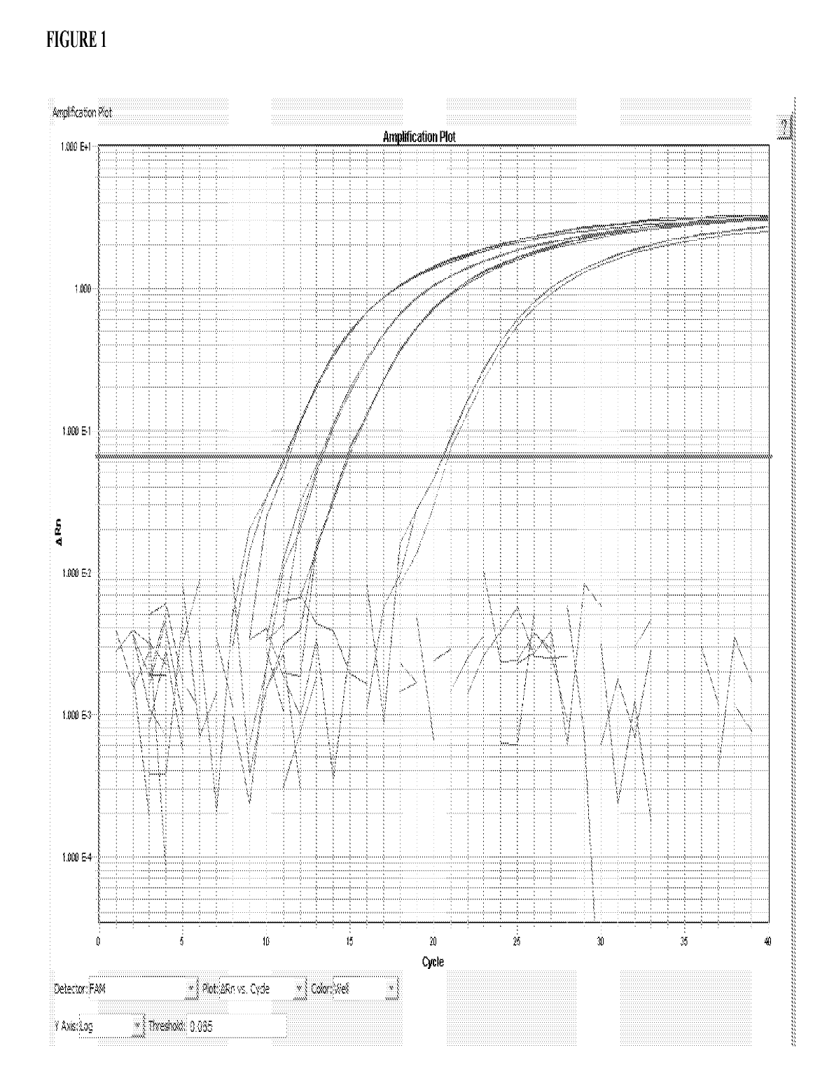

To validate the Burk-16S in a traditional real-time PCR format, 171 DNA samples from B. pseudomallei and B. mallei originating from environmental and clinical sources in Thailand and Australia were run. The forward (Burk16S_Forward: 5′-ATTCTGGCTAATACCCGGAGTG-3′) (SEQ ID NO: 1) and reverse primer (Burk16S_Reverse: 5′-GCAGTTCCCAGGTTGAGCC-3′) (SEQ ID NO: 2) were used in conjunction with a dual labeled probe oligo (Burk16S_Probe: 5′FAM-CAGGCGGTTTGCTAAG-MGB-3′) (SEQ ID NO: 3). The specificity of the assay was examined by running 107 samples of non-B. pseudomallei/non-B. mallei bacterial DNA, including 5 other Burkholderia species. Amplification plots from these validation assays are shown in FIG. 1.

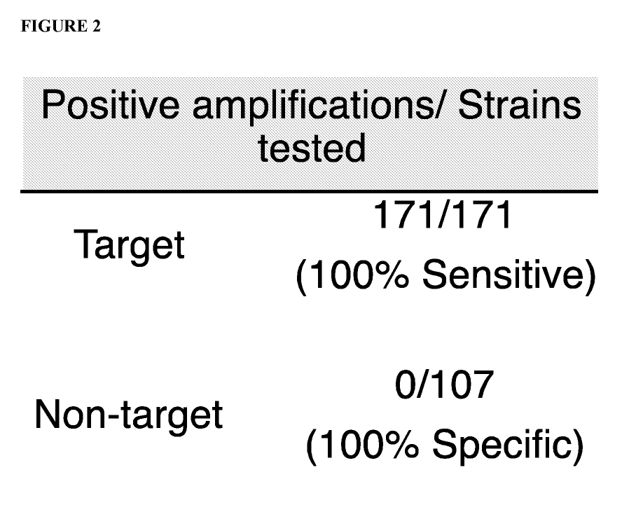

Of the 171 DNA samples from B. pseudomallei and B. mallei, all 171 resulted in positive amplifications while none of the 107 samples of non-B. pseudomallei/non-B. mallei bacterial DNA produced a positive amplification (see the results presented in FIG. 2). The Burk-16S assays run as traditional real-time PCR was found to be 100% sensitive across 171 B. pseudomallei and B. mallei strains, and 100% specific across 107 strains from 43 different organisms.

### Example 2

### Comparison of the Burk-16S Assay to the Conventional TTS1 Assay

The Total RNA Purification Kit (Norgen, Biotek Corporation, Thorold, ON, Canada) was used for the isolation and purification of Burkholderia pseudomallei RNA. One-step real-time reverse transcriptase PCR was performed with the AgPath-ID™ One-Step RT-PCR kit (Life Technologies, Grand Island, N.Y., USA). The forward (Burk16S_Forward: 5′-ATTCTGGCTAATACCCGGAGTG-3′) (SEQ ID NO: 1) and reverse primer (Burk16S_Reverse: 5′-GCAGTTCCCAGGTTGAGCC-3′) (SEQ ID NO: 2) were used in conjunction with a dual labeled probe oligo (Burk16S_Probe: 5′ FAM-CAGGCGGTTTGCTAAG-MGB-3′) (SEQ ID NO: 3).

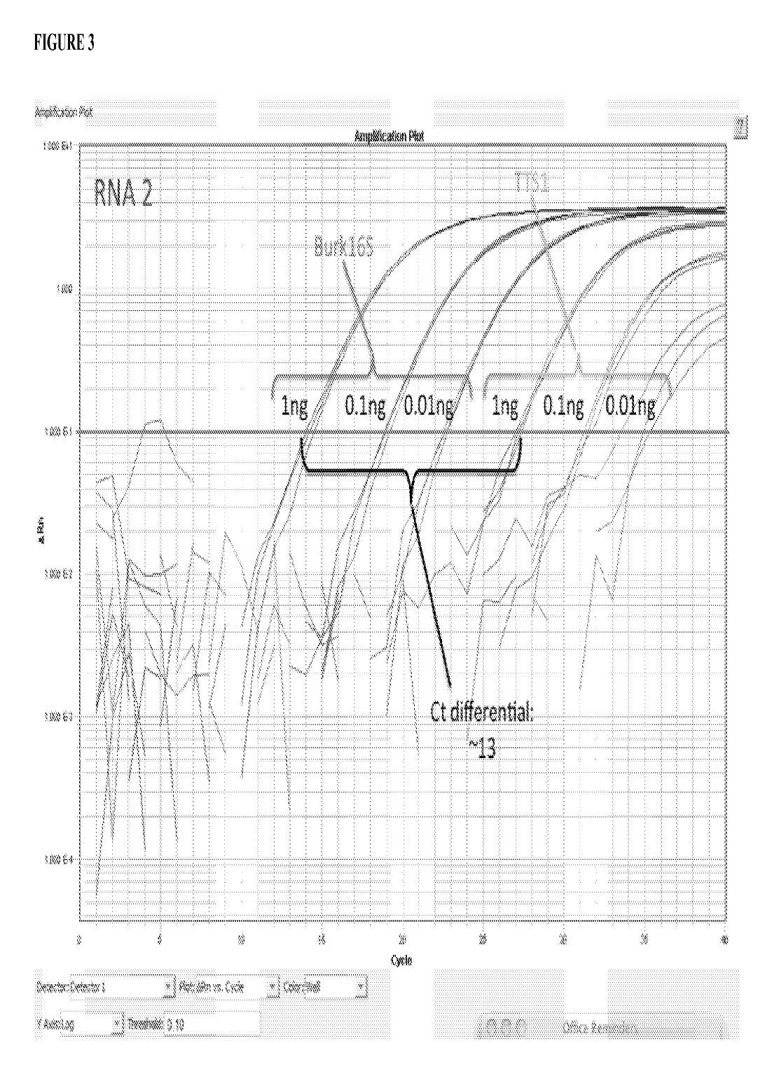

The Burk-16S reverse transcriptase (RT) real-time PCR was evaluated and compared to the highly reliable and previously published real-time assay that targets a Type III Secretion system (TTS1) gene. When both assays were assessed as RT reactions on one nanogram of B. pseudomallei RNA derived from laboratory cultures, the Burk-16S assay consistently amplified from about 10-13 Ct prior to the TTS1 assay, corresponding to an approximate 1000-8,300 fold increase in number of targets present for the Burk-16S assay (see FIG. 3). Cross-reaction or interference of total human RNA was checked independently and with total human RNA spiked into B. pseudomallei RNA. No significant cross-reaction was detected.

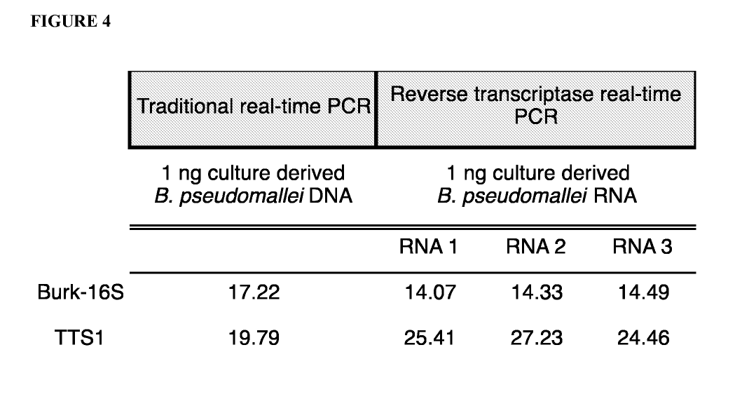

The Burk-16S assay amplified prior than the TTS1 assay in both traditional and reverse transcriptase real-time PCR formats, amplifying approximately 2.5 and between 10 to 13 Ct earlier for traditional and reverse transcriptase configurations, respectively, indicating many more Burk-16S targets than TTS1 genes or transcripts (see representative data in FIG. 4). The early amplification of the Burk-16S assay would increase the likelihood of B. pseudomallei and B. mallei detection in any given sample. It was also found that the Burk-16S assay amplified 1 ng of RNA as a reverse transcriptase real-time PCR around 3 Ct earlier than the same quantity of DNA as a traditional real-time PCR, correlating to about a 10 fold increase of targets in the RNA sample.

A clinical RNA sample extracted from infected aortic tissue using both assays was characterized. The aortic tissue was shipped from Australia without quality assessment. It was found that at a 1000-fold dilution the sample was detected on the Burk-16S RT assay at 27 Ct and on the TTS1 RT assay at 28 Ct. The decrease in differential Ct may represent variation in expression levels of the TTS1 gene in different sample types. It may also be due to the highly degraded nature of the RNA from this sample. However, the Burk-16S target is consistently present in high copy numbers. The use of the Burk-16S RT assay enables faster, more sensitive detection of the organism in a variety of sample types, resulting in more rapid diagnosis and treatment.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

What is claimed is:

1. A pair of isolated oligonucleotides for the amplification of a 16S ribosomal RNA nucleic acid from Burkholderia pseudomallei or Burkholderia mallei consisting of:
a first oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5; and
a second oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5.

2. The pair of isolated oligonucleotides of claim 1, wherein the first oligonucleotide comprises SEQ ID NO: 1 and the second oligonucleotide comprises SEQ ID NO: 2.

3. The pair of isolated oligonucleotides of claim 2, wherein the first oligonucleotide consists of SEQ ID NO: 1 and the second oligonucleotide consists of SEQ ID NO: 2.

4. The pair of isolated oligonucleotides of claim 1, wherein the oligonucleotides produce positive amplifications with nucleic acid samples from B. pseudomallei and B. mallei with at least 80% sensitivity.

5. The pair of isolated oligonucleotides of claim 1, wherein the oligonucleotides do not produce positive amplifications with nucleic acid samples from non-B. pseudomallei and non-B. mallei bacterial species with at least 80% specificity.

6. A method of detecting the presence of B. pseudomallei , B. mallei, or both in a sample, the method comprising:
a) contacting the sample with the pair of oligonucleotides of claim 1 under conditions whereby amplification of the 16S ribosomal RNA nucleic acid can occur; and
b) detecting the amplified 16S ribosomal RNA nucleic acid.

7. The method of claim 6, wherein the first oligonucleotide comprises SEQ ID NO: 1 and the second oligonucleotide comprises SEQ ID NO: 2.

8. The method according to claim 6, wherein detecting the amplified nucleic acid comprises contacting the amplified 16S ribosomal RNA nucleic acid with an oligonucleotide probe under conditions whereby hybridization can occur, wherein the oligonucleotide probe comprises a flourophore and/or a quencher.

9. The method according to claim 8, wherein the oligonucleotide probe comprises a nucleotide sequence of SEQ ID NO: 3.

10. The method of claim 6, wherein the amplification produces a cDNA of the 16S ribosomal RNA nucleic acid with reverse-transcriptase PCR.

11. The method of claim 6, wherein the amplification is accomplished with quantitative real-time PCR.

12. The method of claim 11, further comprising using a dye selected from the group consisting of: SYBR GREEN, 6-FAM, HEX, JOE, ROX, TET, CY3, CY5, TAMRA, TEXAS RED in the quantitative real-time PCR.

13. The method of claim 6, wherein the amplified 16S ribosomal RNA nucleic acid is specific to B. pseudomallei and/or B. mallei and detection of the amplified 16S ribosomal RNA nucleic acid confirms the presence of B. pseudomallei and/or B. mallei in the sample.

14. A diagnostic kit for detecting the presence of B. pseudomallei, B. mallei, or both in a sample, the kit comprising:
a first oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5;
a second oligonucleotide of between 15 and 30 nucleotides in length and comprising at least 15 contiguous nucleotides of a nucleotide sequence selected from the group consisting of SEQ ID NO: 4, SEQ ID NO: 5, the reverse complementary nucleotide sequence of SEQ ID NO: 4, and the reverse complementary nucleotide sequence of SEQ ID NO: 5; and
amplification reagents.

15. The diagnostic kit of claim 14, wherein the first oligonucleotide comprises SEQ ID NO: 1 and the second oligonucleotide comprises SEQ ID NO: 2.

16. The diagnostic kit of claim 14, further comprising an oligonucleotide probe comprising a flourophore and/or a quencher.

17. The diagnostic kit of claim 16, wherein the oligonucleotide probe comprises a nucleotide sequence of SEQ ID NO: 3.
